(19) United States
(12) Patent Application Publication
(10) Pub. No.: US 2019/0282393 A1
(43) Pub. Date: Sep. 19, 2019

(54) CRYOGENIC CHAMBER FOR A HORSE OR A CAMEL

(71) Applicant: Robert Klejdysz, Warsaw (PL)

(72) Inventor: Robert Klejdysz, Warsaw (PL)

(21) Appl. No.: 16/464,687

(22) PCT Filed: Dec. 11, 2017
(86) PCT No.: PCT/PL2017/000123

§ 371 Date: May 29, 2019

(30) Foreign Application Priority Data
Dec. 11, 2016 (PL) P.419764

(51) Int. Cl. A61F 7/00; A61D 11/00; A61D 7/00; A61H 33/00

(52) U.S. Cl. CPC A61F 7/0053; A61D 11/00; A61D 7/00; A61H 2203/03; A61F 2007/0068; A61H 2201/0214; A61H 33/6021

(57) ABSTRACT

The cryogenic chamber has a treatment cabin (12) cooled by a cooling factor in the form of liquid air or liquid nitrogen distributed through the nozzle system (17). The treatment cabin (12), which can be open at the top or equipped with a roof, is available in a stationary or mobile version, namely it can be placed on a trailer and is transitional, namely it has a rear door (2) and a front door (1) and its side walls have outlet openings (8, 11) and a grid (7) intended for introducing the cooling factor from the cooling chamber (16), whereas in the treatment cabin (12) at the front and at the back there are elements (18) blocking the animal taking the treatment. The shape of the treatment cabin (12) is based on a rectangle. The chamber is equipped with roller blinds (4, 5) and its operation is controlled by a panel with a monitor as well as a power supply and control system (3).

The subject matter of the invention is a systemic cryotherapy chamber for a horse or a camel.

Systemic cryotherapy is used as an incentive and stimulating application in the cryogenic chamber at a temperature below −120° C. in a short period of time. It works on the whole-body surface to evoke reflexes and defensive reactions that act therapeutically and effectively restore the balance of the body. This method is used in neurosurgical disorders with muscle spasticity, in all post-traumatic and post-operative conditions as well as primary and secondary degenerative changes affecting the motor apparatus. Extremely effective treatment effects can be observed in sport traumatology. Systemic cryotherapy has become the method of treatment choice for the physical sport injuries and biological regeneration method.

A cold surface treatment method is also used at veterinary clinics. Lowering the temperature of tissues reduces or relieves pain, inhibits inflammation, reduces or resolves edema. Cryotherapy is indicated for disorders of the movement apparatus, edema after fractures and dislocations, acute tendonitis, joint capsules and muscles inflammation, post-traumatic joint contractures, nervous system diseases and rheumatic diseases.

A cryogenic chamber for a horse is known from the Polish description of the invention P.414791, which consists of a main treatment cabin located on a base, the shape of which is preferably based on a hexagon formed of a rectangle in which a shorter side is adjoined to a longer isosceles trapezoid base, that allows placing the whole device in a trailer intended for transportation of horses. The main cabin preferably having its shape based on rectangle is provided with a floor and walls preferably perpendicular to the floor and open at the top. The back wall of the treatment cabin is a door, preferably a double-leaf door, sealing the main cabin. The height of all walls of the main cabin and the rear door is from 140 cm up to 150 cm to cool the horse up to the height of its shoulders. The floor of the treatment cabin is made of a high durability material. There are shields attached from the top to the side walls of the cabin which limit the horse's movement during the treatment and protect the service against biting. A raised blocking element is attached to the front wall to block the horse to avoid undesirable movements during the procedure. In front of the front wall of the treatment cabin there is a cooling chamber in which the liquid nitrogen evaporates and the air is cooled, which is next delivered to the treatment cabin through the air inlet located in the front wall of the treatment cabin. The cooled air inlet is equipped with a cover shaped so that the cold air is not blown directly on the horse taking the treatment but flows around the cabin. The door closing the main cabin is equipped with a slide bolt lock. The side walls of the treatment cabin are made of material resistant to low temperatures and resistant to mechanical impact. The blocking element of the horse is covered with a covering protecting the horse against low-temperature air burns. On the outer side, there is a platform at each side wall of the main cabin. On one side of the treatment cabin there is a power cabinet equipped with a panel control. On the opposite side, there is the door of the cooling chamber. There is a technical room behind the cooling chamber. The control panel is preferably equipped with an audio system that plays music during the procedure to drown out the sound of operating devices without frightening the horse.

The disadvantage of the presented solution is the design of the chamber, in which some solutions were applied making the treatment difficult. This applies to the blocking elements of the horse taking the treatment and the cryogenic chamber having only the rear door. This solution makes it difficult to lead out the treated horse, which additionally increases its stress.

The cryogenic chamber which is the subject matter of this invention has a treatment cabin cooled by a cooling factor in the form of liquid air or liquid nitrogen distributed through a system of nozzles. The shape of the treatment cabin is based on a rectangle. The treatment cabin is transitional i.e. it is equipped with a front door and a rear door, wherein the rear door being an entrance door through which the treated animal is led in and the front door being an exit door through which the treated animal is led out. Each door is a single-leaf or a double-leaf door and it is closed automatically by the actuator or by hand. Each door is equipped with a slide bolt lock to protect against undesirable opening. There are animal blocking elements in the treatment cabin that prevent the animal from forcing the door. The doors and side walls of the treatment cabin are preferably perpendicular to the floor. The treatment cabin is open from the top or closed from the top by a roof. The width of the cabin is set to limit undesirable sideways movements of the animal taking the treatment. The height of the treatment cabin walls and doors is preferably from 140 cm up to 180 cm for a horse and from 160 cm up to 230 cm for a camel. In the side wall of the cryogenic chamber there is a cooling chamber equipped with nozzles from which the cooling factor is forced through the outlet grid located on the side wall of the treatment cabin. Preferably, the outlet grid is located at the center of the side wall so that the cooling factor is blown between the forelimbs and hindlimbs of the animal. The walls of the treatment cabin are equipped with bottom outlet openings and upper outlet openings. The bottom outlet openings are used to clean the inside of the treatment cabin after the procedure. The upper outlet openings with a cover are used to collect excess steam generated in the treatment cabin. In the treatment cabin, there are two elements blocking the treated animal and protecting it against contact with cooled metal elements. One of the elements blocks the animal at the front and the other at the back. The elements are in the form of rods, which are placed in sockets located on the side walls of the treatment cabin. The blocking elements are covered with a covering protecting the animal against burning. The walls and doors of the treatment cabin are made of a material resistant to low temperatures and resistant to mechanical impact and its floor is preferably made of silicone. In the cryogenic chamber, outside of the treatment cabin there is a movable boom with a food container holder for the animal. In the cryogenic chamber, in addition to the treatment cabin, there is a power cabinet with a monitor and a power supply and control system preferably located in front of the cryogenic chamber. The floor of the cryogenic chamber is made of silicone characterized by high mechanical strength and resistance to low temperatures. The cryogenic chamber has front and rear roller blinds protecting against draft and blowing steam from the treatment cabin. The cryogenic chamber is equipped with an audio system that plays music that drowns out the sounds of operating devices, preventing the animal from being frightened. The work of the cryogenic chamber can, for safety reasons, be interrupted by means of an emergency switch. The cryogenic chamber may be a stationary or mobile device adapted to be placed on a trailer. In the mobile version of the chamber, the chassis is pulled out from under the trailer so that the front gangway of the trailer can be opened and the animal taking the treatment can be led out from the treatment cabin.

The solution according to the invention is characterized by a construction that makes the treatment less stressful for animals taking the treatment. These solutions include the use of the front door to the animal exit from the cabin as well as equipment for animal feeding. Music that drowns out the sounds of operating devices also reduces the stress of the animal.

In the following, the invention will be described in more detail by means of embodiment examples and with reference to the enclosed drawings.

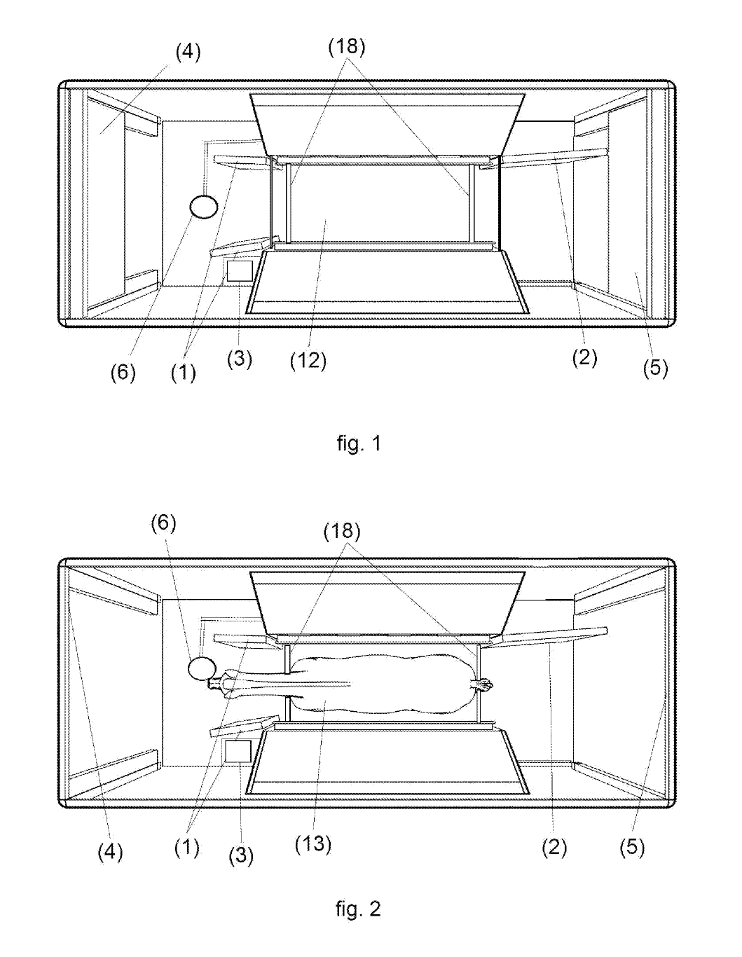

FIG. 1 shows a cryogenic chamber in a top view,

FIG. 2 shows a cryogenic chamber with a horse in a treatment cabin in a top view,

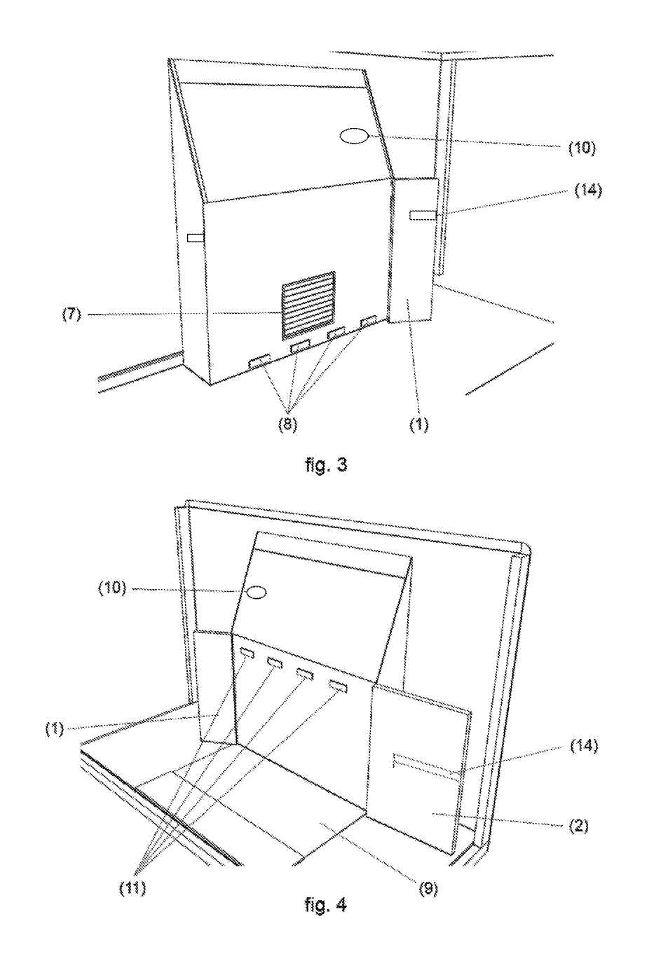

FIG. 3 shows the side wall of the cabin with cold air outlet grid and bottom outlet openings in an axiomatic view,

FIG. 4 shows the wall of the cabin with the upper outlet openings and the loudspeaker of the audio system in an axiomatic view,

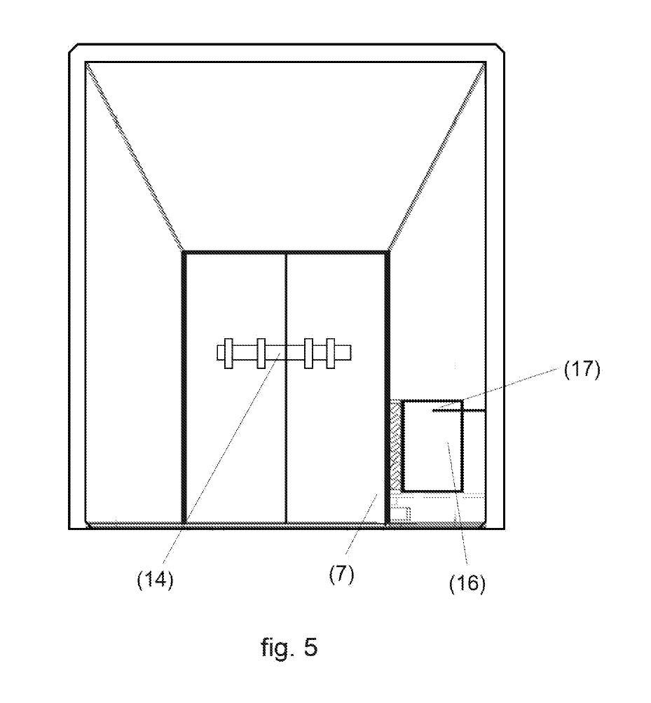

FIG. 5 shows the door closed with a slide bolt lock and the cooling chamber with nozzles in a rear view.

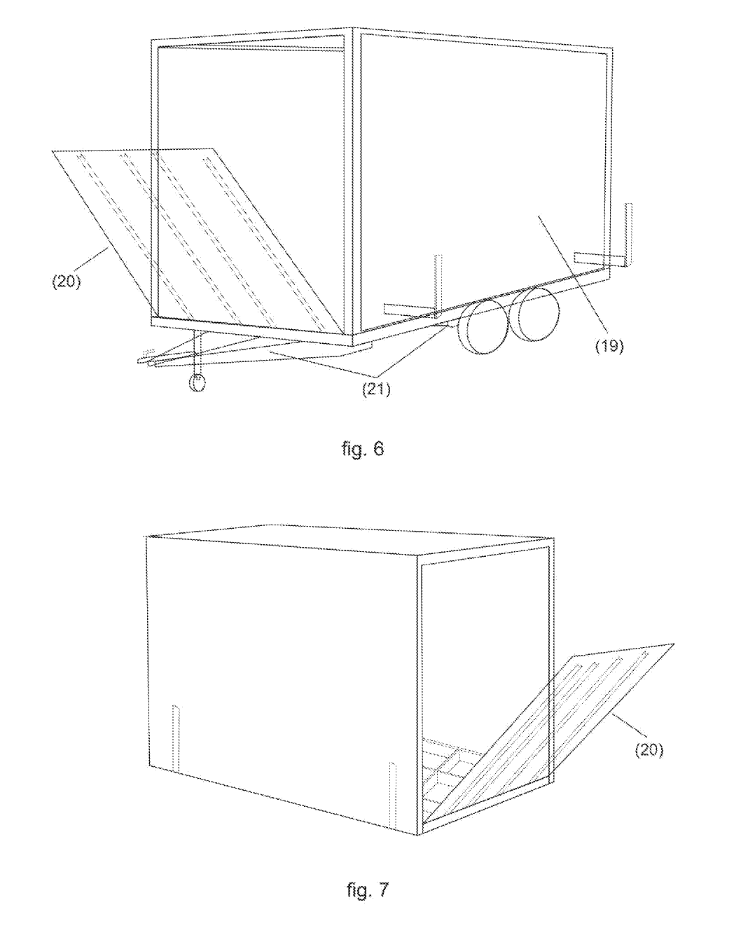

FIG. 6 and FIG. 7 shows the trailer of the mobile version of the cryogenic chamber in an axiomatic view.

### EXAMPLE

The cryogenic chamber has a treatment cabin (12) cooled by a cooling factor in the form of liquid air or liquid nitrogen distributed through a system of nozzles (17). The cryogenic chamber includes the treatment cabin (12) in the shape of a cuboid open at the top. The animal (13) taking the treatment has a head outside the treatment cabin (12) at an ambient temperature. The treatment cabin (12) is transitional i.e. it is equipped with a front door (1) and a rear door (2), wherein the rear door (2) being an entrance door through which the treated animal (13) is led in and the front door (1) being an exit door through which the treated animal (13) is led out. The front door (1) is a double-leaf door and the rear door (2) is a single-leaf door. The doors (1, 2) are closed automatically by the actuator or by hand. The doors are equipped with a slide bolt lock (14) protecting against undesirable opening, which prevents the animal (13) from forcing it. The width of the cabin is set to limit undesirable sideways movements of the animal taking the treatment and ranges from 70 cm up to 100 cm depending on the animal (13). The height of the walls and the doors of the treatment cabin is from 140 cm up to 230 cm depending on the animal (13). The treatment cabin is equipped with two elements (18) blocking the animal (13) taking the treatment to prevent forcing the doors (1, 2) by the animal (13). The blocking elements are made of metal and are covered with silicone to protect the animal against contact with cooled metal elements. One of the elements (18) blocks the animal at the front and the other at the back. The elements (18) are in the form of rods, which are placed in sockets located on the side walls of the treatment cabin (12). The blocking elements (18) are covered with a silicone covering protecting the animal against burning. In the side wall of the treatment cabin (12) there is a cooling chamber (16) equipped with nozzles (17) from which cold air is forced through the outlet grid (7) located on the side wall of the treatment cabin (12). Preferably the outlet grid (7) is located at the center of the side wall so that the cold air is blown between the forelimbs and hindlimbs of the horse (13). The treatment cabin (12) is equipped with bottom outlet openings (8) and upper outlet openings (11). The bottom outlet openings (8) are used to clean the inside of the treatment cabin (12) after the procedure. The upper outlet openings (11) with a cover are used to collect excess steam generated in the treatment cabin (12). In the cryogenic chamber, outside treatment cabin (12) there is a movable boom (6) with a holder for the food container for the horse (13). In the cryogenic chamber, in addition to the treatment cabin (12), there is a power cabinet with a monitor and a power supply and control system (3) preferably located in front of the cryogenic chamber. The floor (9) of the treatment cabin (12) is made of silicone characterized by high mechanical strength and resistance to low temperatures. The walls and the doors of the treatment cabin (12) are made of a material resistant to low temperatures and resistant to mechanical impact. The cryogenic chamber is equipped with a front (4) and a rear (5) roller blinds protecting against draft and blowing steam from the treatment cabin (12.) The cryogenic chamber is equipped with an audio system (10) that plays music that drowns out the sounds of operating devices, preventing the animal (13) from being frightened. The work of the cryogenic chamber can, for safety reasons, be interrupted by means of an emergency switch. In the mobile version of the chamber, the chassis (20) is pulled out from under the trailer (19) so that the front gangway (21) of the trailer (19) can be opened and the animal (13) can be led out at the front from the treatment cabin (12).

1. A cryogenic chamber for a horse or a camel equipped with a treatment cabin, to which through a system of nozzles the cooling factor in the form of liquid air or liquid nitrogen is supplied from the cooling chamber through an inlet located in the wall of the treatment cabin whose operation is controlled by the power supply and control system, wherein the shape of the said treatment cabin being preferably based on a rectangle while its walls being preferably perpendicular to the floor and its rear wall being a door equipped with a slide bolt lock, whereas the floor of the treatment cabin being made of a high durability material and the side walls being made of resistant material for low temperatures and mechanical impacts, and besides, the said treatment cabin being equipped with an element with a cover blocking the animal taking the treatment and additionally, the said treatment cabin being equipped with an audio system that plays music drowning out the sounds of operating devices, characterized in that being available in a stationary or mobile version in which it is located on a trailer while the said treatment cabin (12), which can be open from the top or equipped with a roof is transitional as well as having outlet openings (8, 11) located in its side walls and an outlet grid for introducing the cooling factor from the cooling chamber (16) located in the side wall of the cryogenic chamber whereas the elements (18) blocking the animal (13) taking the treatment are located at the front and rear of the said treatment cabin (12), additionally the chamber is equipped with roller blinds (4, 5), a monitor with the power supply and control system (3) as well as the animal feeding device for the animal (13) taking the treatment.

2. The chamber according to claim 1, characterized in that the transitional treatment cabin is equipped with a rear door (2) for leading in the animal (13) taking the treatment to the treatment cabin (12) and a front door (1) for leading out the animal (13) after the treatment.

3. The chamber according to claim 1, characterized in that the width of the treatment cabin (12) preferably ranges from 70 cm up to 100 cm limiting the movements of the animal (13) taking the treatment from side to side thus it limits the undesired movements of the animal (13) during the procedure.

4. The chamber according to claim 1, characterized in that the elements (18) blocking the animal taking the treatment and protecting it from contact with the cooled metal elements are in the form of rods in a silicone cover, which are placed in sockets on the side walls of the treatment cabin (12).

5. The chamber according to claim 1, characterized in that the height of the side walls as well as the front (1) and the rear (2) doors of the treatment cabin (12) preferably is from 140 cm to 180 cm in the chamber intended for the horse.

6. The chamber according to claim 1, characterized in that the height of the side walls as well as the front (1) and the rear (2) doors of the treatment cabin (12) preferably is from 160 cm to 230 cm in the chamber intended for the camel.

7. The chamber according to claim 1, characterized in that in the side wall there is a cooling chamber (16) from which through the outlet grid (7) the cryogenic factor by means of the nozzle system (17) is forced to the treatment cabin (12) whereas the outlet grid (7) is preferably located in the middle of the side wall, so that the cryogenic factor is introduced between the forelimbs and hindlimbs of the animal taking the treatment.

8. The chamber according to claim 1, characterized in that the treatment cabin (12) has post-treatment bottom outlet openings (8) of cold air used after the end of the treatment to clean the interior of the treatment cabin (12).

9. The chamber according to claim 1, characterized in that the treatment cabin (12) has upper outlet openings (11) with a cover to collect cold air vapor excess during the treatment.

10. The chamber according to claim 1, characterized in that the front door (1) and the rear door (2) of the treatment cabin (12) are single-leaf or double-leaf.

11. The chamber according to claim 1, characterized in that the doors (1, 2) of the treatment cabin (12) are closed automatically by the actuator or by hand.

12. The chamber according to claim 1, characterized in that the floor (9) of the treatment cabin is preferably made of silicone resistant to low temperature.

13. The chamber according to claim 1, characterized in that a movable boom (6) is mounted in front of the treatment cabin (12) with a holder for the food container for the animal (13).

14. The chamber according to claim 1, characterized in that it is equipped with front (4) and rear (5) roller blinds protecting against draft and blowing steam from the treatment cabin (12).

15. The chamber according to claim 1, characterized in that the monitor with the power supply and control system (3) is placed at the front of the chamber.

16. The chamber according to claim 1, characterized in that it is equipped with an emergency switch.

17. The chamber according to claim 1, characterized in that in the mobile version of the chamber, the chassis (20) is pulled out from under the trailer (19) so that the front gangway (21) of the trailer (19) can be opened and the animal (13) can be led out at the front from the treatment cabin (12).
